(19) United States
(12) Patent Application Publication
(10) Pub. No.: US 2025/0352689 A1
(43) Pub. Date: Nov. 20, 2025

(54) Method for Preparing an Antibacterial Polyester and Method for Producing an Antibacterial Fiber

(71) Applicant: Shanghai JIECON Chemicals Hi-tech Co., Ltd., Shanghai (CN)

(72) Inventors: Hongxia DONG, Shanghai (CN); Shihan HE, Shanghai (CN); Zhengsheng MA, Shanghai (CN); Wenxiu YU, Shanghai (CN); Yifan MA, Shanghai (CN)

(73) Assignee: Shanghai JIECON Chemicals Hi-tech Co., Ltd., Shanghai (CN)

(21) Appl. No.: 19/283,278

(22) Filed: Jul. 29, 2025

Related U.S. Application Data
(63) Continuation of application No. PCT/CN2024/121777, filed on Sep. 27, 2024.

(30) Foreign Application Priority Data
Oct. 18, 2023 (CN) 202311349929.0

(51) Int. Cl. A61L 15/26; A01N 41/04; A01P 1/00; A61L 15/44; C08G 63/688

(52) U.S. Cl. CPC A61L 15/26; A01N 41/04; A01P 1/00; A61L 15/44; C08G 63/6886

(57) ABSTRACT

A preparation method for an antibacterial polyester, an antibacterial polyester obtained by the preparation method, a production method for an antibacterial fiber, an antibacterial fiber, and the use of the antibacterial fiber in producing medical surgical article. The invention primarily addresses the poor wash durability and antibacterial performance of existing antibacterial polyester fibers. The method for preparing antibacterial polyesters comprises: (i) performing an esterification reaction between a diol and a dibasic acid to obtain Esterified Material I; (ii) mixing Esterified Material I with a reactive antibacterial component, and conducting a pre-polycondensation reaction to obtain a pre-polycondensate; (iii) performing a final polycondensation reaction on the pre-polycondensate to obtain the antibacterial polyester. The reactive antibacterial component is obtained by esterification of a compound represented by formula (3) with a glycol, wherein: Q represents a quaternary ammonium group containing a C6-C20 long-chain hydrocarbon group; Ar represents an aromatic ring.

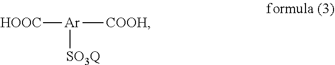

### TECHNICAL FIELD

The present invention relates to a preparation method for an antibacterial polyester, an antibacterial polyester obtained by the preparation method, a production method for an antibacterial fiber, an antibacterial fiber, and the use of the antibacterial fiber in producing medical surgical article.

### BACKGROUND TECHNOLOGY

Polyester fiber is one of the most widely produced and utilized synthetic fibers, with broad applications in home textiles, apparel, and industrial textiles. In 2022, the total polyester fiber production in China exceeded 50 million tons.

Bacterial infections remain one of the major threats to human health. The development of antibacterial polyester fibers is of great significance, as it can help block bacterial transmission, reduce the risk of infection, minimize the use of antibiotics, and improve public health.

At present, antibacterial polyester fibers are mainly prepared by physically blending inorganic antibacterial agents such as nanosilver or nano-zinc oxide. However, the antibacterial effect of such fibers relies on the diffusion and release of the antibacterial agents, and diminishes once the agents are depleted. This is particularly problematic during routine washing, where the agents are often washed away, resulting in poor durability of antibacterial properties.

In addition, inorganic antibacterial agents often exhibit high biological toxicity, which may cause skin allergy when used in textiles worn close to the body, thereby limiting their application.

The current large-scale commercial synthesis of polyester fibers typically involves melt polycondensation of dicarboxylic acids and diols, followed by melt spinning. Incorporating eco-friendly organic antibacterial agents into the polyester backbone through copolymerization introduces covalent bonding of the agents into the polymer chain. This approach not only imparts long-lasting antibacterial properties to the polyester but also effectively reduces the biological toxicity of the antibacterial agents. As such, it represents a promising direction for the development of antibacterial polyester fibers.

### SUMMARY OF THE INVENTION

One of the technical problems addressed by the present invention is the poor wash durability of antibacterial performance in existing antibacterial polyester fibers. The invention provides a new method for preparing antibacterial polyester, whereby the antibacterial polyester fiber produced using the antibacterial polyester improved wash-resistant antibacterial properties.

To solve the above technical problem, the technical solution of the present invention is as follows:

A method for preparing antibacterial polyester, comprising the following steps:
Step (i) performing an esterification reaction between a diol and a dibasic acid to obtain Esterified Material I;
Step (ii) mixing the Esterified Material I with a reactive antibacterial component and performing a pre-polycondensation reaction to obtain a pre-polycondensate;
Step (iii) performing a final polycondensation reaction on the pre-polycondensate to obtain an antibacterial polyester;

The reactive antibacterial component is obtained by esterification between a compound represented by Formula (3) and a glycol, wherein:

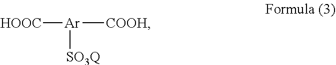

Q is a quaternary ammonium group containing a long-chain hydrocarbon group containing 6 to 20 carbon atoms; Ar represents an aromatic ring.

A key aspect of the invention lies in the use of the reactive antibacterial component during the preparation of the antibacterial polyester. Once this reactive antibacterial component is disclosed, a person skilled in the art can reasonably select process conditions to achieve comparable technical effects without exercising inventive skill when applying the component to prepare antibacterial polyester. However, in comparison with direct co-esterification of the component represented by Formula (3) in step (i), or adding the reactive antibacterial component during step (i) for esterification, it has been found that adding the reactive antibacterial component during the pre-polycondensation stage yields antibacterial polyester with significantly better antibacterial performance than when the component is added during esterification.

In the above technical solution, Ar is preferably a phenyl ring or a naphthyl ring.

In the above technical solution, in step (i), the diol is preferably selected from at least one of ethylene glycol, 1,3-propanediol, 1,4-butanediol, 1,5-pentanediol, and 1,4-cyclohexanedimethanol. For comparison purposes only, ethylene glycol is commonly used in the embodiments of the invention.

In the above technical solution, in step (i), the dibasic acid is preferably selected from at least one of terephthalic acid, succinic acid, adipic acid, isophthalic acid, and furan dicarboxylic acid. For comparison purposes only, terephthalic acid is commonly used in the embodiments of the invention.

In the above technical solution, in step (i), a molar ratio of the diol to the dibasic acid is preferably from 1.1 to 1.5. For example and without limitation, the molar ratio may be 1.15, 1.2, 1.25, 1.3, 1.35, 1.4, 1.45, etc.

In the above technical solution, a degree of esterification in step (i) is preferably 95% to 99%, for example and without limitation, 95.5%, 96%, 96.5%, 97%, 97.5%, 98%, 98.5%, etc.

To achieve the above degree of esterification, those skilled in the art may reasonably select esterification temperature and time based on actual conditions such as the capabilities of the reaction equipment. As a general principle, higher esterification temperatures and longer esterification times favor higher degree of esterifications.

As a non-limiting example, an esterification temperature in step (i) may be selected from 150° C. to 250° C. More specifically and non-limited examples include 160° C., 170° C., 180° C., 190° C., 200° C., 210° C., 220° C., 230° C., 240° C., etc. Within this temperature range, when water produced during the esterification reaction is continuously removed, the target degree of esterification can typically be achieved within 0.5 to 2 hours, even without using a catalyst, as in the embodiments of this invention. Of course, an esterification catalyst may be used to accelerate the reaction, and the esterification reaction is faster when an esterification catalyst is employed.

The esterification reaction of step (i) may be conducted under self-generated pressure or under elevated pressure by introducing an inert gas, such as nitrogen. Since the esterification in step (i) is a liquid-phase reaction, pressure exerts no significant influence on the reaction process.

In the above technical solution, in step (ii), a mass ratio of the reactive antibacterial component (based on N content) to Esterified Material I (based on dibasic acid required for its preparation) is preferably t:100, where t is greater than 0 and less than or equal to 4. For example and without limitation, t may be 0.01, 0.05, 0.1, 0.2, 0.3, 0.4, 0.5, 0.6, 0.7, 0.8, 0.9, 1, 1.5, 2, 2.5, 3, 3.5, etc.

In the above technical solution, in step (ii), a reaction pressure is preferably from 400 to 600 Pa, for example and without limitation, 410 Pa, 420 Pa, 430 Pa, 440 Pa, 450 Pa, 460 Pa, 470 Pa, 480 Pa, 490 Pa, 500 Pa, 510 Pa, 520 Pa, 530 Pa, 540 Pa, 550 Pa, etc.

In the above technical solution, in step (ii), a reaction temperature is preferably from 255° C. to 265° C., for example and without limitation, 256° C., 257° C., 258° C., 259° C., 260° C., 261° C., 262° C., 263° C., 264° C., etc.

In the above technical solution, in step (ii), a reaction time is preferably from 30 to 60 minutes, for example and without limitation, 35 minutes, 40 minutes, 45 minutes, 50 minutes, 55 minutes, etc.

In the above technical solution, in step (iii), a reaction pressure is preferably less than or equal to 100 Pa, for example and without limitation, 5 Pa, 10 Pa, 20 Pa, 30 Pa, 40 Pa, 50 Pa, 60 Pa, 70 Pa, 80 Pa, 90 Pa, etc.

In the above technical solution, in step (iii), a reaction temperature is preferably from 270° C. to 285° C., for example and without limitation, 271° C., 272° C., 273° C., 274° C., 275° C., 276° C., 277° C., 278° C., 279° C., 280° C., 281° C., 282° C., 283° C., 284° C., etc.

It is known in the art that reducing the reaction pressure and increasing the reaction temperature in step (iii) facilitates the final polycondensation reaction and helps improve the intrinsic viscosity of the resulting product. Once the reaction pressure and temperature in step (iii) are fixed, the intrinsic viscosity tends to increase with the reaction time. Preferably, in the above technical solution, step (iii) is carried out until the intrinsic viscosity reaches 0.60˜0.75 dl/g. For comparison purposes only, the embodiments and comparative embodiments all reached 0.68 dl/g.

In the above technical solution, under the specified reaction temperature and pressure conditions in step (iii), the reaction time of approximately 1.5 to 3.0 hours is generally sufficient to achieve the desired range of the intrinsic viscosity.

In the above technical solution, the synthesis method of the reactive antibacterial component comprises the following steps:
(1) Subjecting Compound 1 and Compound 2 to an ion-exchange reaction in a solvent to obtain Intermediate Compound 3;

Compound 1 conforms to the structure represented by Formula (1):

Q X⁻, Formula (1);

Compound 2 conforms to the structure represented by Formula (2):

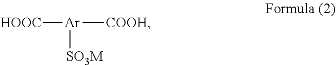

Intermediate Compound 3 conforms to the structure represented by Formula (3);

X is Cl or Br; M is an alkali metal;
(2) Subjecting Intermediate Compound 3 to an esterification reaction with a glycol to obtain the reactive antibacterial component.

In the above technical solution, the solvent in step (1) is preferably water.

The ion-exchange reaction in step (1) may be represented by the following reaction

equation:

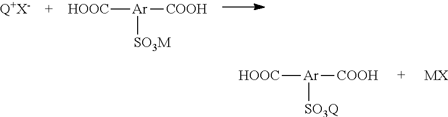

When water is used as the solvent for the ion-exchange reaction, Intermediate Compound 3 precipitates from the reaction system, which facilitates its separation from the reaction mixture.

Non-limiting examples of Compound 1 include, but are not limited to: benzalkonium chloride, hexyltrimethylammonium chloride, octyltrimethylammonium chloride, decyltrimethylammonium chloride, dodecyltrimethylammonium chloride, tetradecyltrimethylammonium chloride, hexadecyltrimethylammonium chloride, octadecyltrimethylammonium chloride, N-hexylpyridinium chloride, N-octylpyridinium chloride, N-decylpyridinium chloride, N-dodecylpyridinium chloride, N-tetradecylpyridinium chloride, N-hexadecylpyridinium chloride, N-octadecylpyridinium chloride, 1-hexyl-3-methylimidazolium bromide, 1-octyl-3-methylimidazolium bromide, 1-decyl-3-methylimidazolium bromide, 1-dodecyl-3-methylimidazolium bromide, 1-tetradecyl-3-methylimidazolium bromide, 1-hexadecyl-3-methylimidazolium bromide, 1-octadecyl-3-methylimidazolium bromide, dimethyldihexylammonium Cholride, dimethyldioctylammonium cholride, didecyldimethylammonium chloride, didodecyldimethylammonium chloride, ditetradecyldimethylammonium chloride, dihexadecyldimethylammonium chloride or dioctadecyldimethylammonium chloride.

In the above technical solution, an optional form of Compound 1 conforms to the structure represented by Formula (1a):

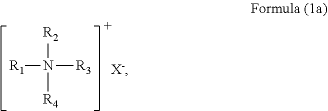

R₁ is a long-chain hydrocarbon group containing 6 to 20 carbon atoms;

R₂˜R₄ are short-chain hydrocarbon groups, preferably independently selected from C₁˜C₂ alkyl groups.

As non-limiting examples, number of carbon atoms in R₁ may be, but is not limited to, 7, 8, 9, 10, 11, 12, 13, 14, 15, 16, 17, 18, or 19, etc. R₁ may be an alkyl, alkenyl, or aryl group.

In the above technical solution, Compound 2 preferably conforms to the structure represented by Formula (2a):

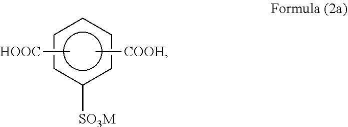

As an example, the Compound 2 used in the embodiments of the present invention is meta-phthalic acid-5-sulfonic acid alkali metal salt.

In the above technical solution, the ion exchange reaction proceeds rapidly and completely, similar to the exchange reactions of inorganic ions. Therefore, there are no particular restrictions on the specific process conditions of the ion exchange reaction, and conventional process conditions known in the art can be used. For example, Compound 1 may be added to the reaction system as an aqueous solution with a weight concentration of 1˜10% (for example, but not limited to, 1%, 2%, 3%, 4%, 5%, 6%, 7%, 8%, 9%, 10%, etc.). Likewise, Compound 2 may be added as an aqueous solution with a weight concentration of 1˜10% (for example, but not limited to, 1%, 2%, 3%, 4%, 5%, 6%, 7%, 8%, 9%, 10%, etc.). The reaction temperature is not particularly restricted and can be carried out at room temperature. There are also no particular limitations on reaction time. Since the ion exchange reaction itself occurs instantaneously, extending the reaction holding time within a certain range, i.e., the apparent reaction time, helps obtain a better precipitated form of the product, facilitating washing and separation. For instance, the apparent reaction time may be 1.5˜3 hours. In practice, the apparent reaction time mainly refers to the aging time of the ion exchange product in the system because ion exchange reaction itself occurs instantaneously.

In the above technical solution, the glycol in step (2) is preferably selected from at least one of the following: 1,3-propanediol, 1,4-butanediol, 1,5-pentanediol, 1,4-cyclohexanedimethanol, and glycols based on OCH₂CH₂ units. The glycols based on OCH₂CH₂ units conform to Formula (4):

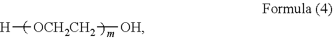

A number-average molecular weight of OCH₂CH₂ based glycols is preferably more than or equal to that of ethylene glycol and less than or equal to 4000 g/mol, with m being a value corresponding to the required molecular weight.

Examples of such OCH₂CH₂ based glycols include, but are not limited to: ethylene glycol, diethylene glycol, triethylene glycol, tetraethylene glycol, polyethylene glycol 300, polyethylene glycol 400, polyethylene glycol 500, polyethylene glycol 600, polyethylene glycol 800, polyethylene glycol 1000, polyethylene glycol 1500, polyethylene glycol 2000, polyethylene glycol 2500, polyethylene glycol 3000, polyethylene glycol 3500, polyethylene glycol 4000, etc. According to naming conventions of polyethylene glycol, the number following “polyethylene glycol” indicates its number-average molecular weight.

We have found that the choice of glycol used in step (2) significantly affects the antibacterial properties of the antibacterial polyester. When 1,4-cyclohexanedimethanol is used, the antibacterial performance is notably superior compared to using ethylene glycol, 1,3-propanediol, 1,4-butanediol, or polyethylene glycol.

We have further found that when a mixture of ethylene glycol and 1,4-cyclohexanedimethanol is used in step (2), a synergistic effect is achieved in enhancing the antibacterial properties of the antibacterial polyester. There is no strict limitation on the ratio between the ethylene glycol and 1,4-cyclohexanedimethanol, as comparable synergistic effects can be achieved across a broad range. As a non-limiting example, a molar ratio of ethylene glycol to 1,4-cyclohexanedimethanol is from 0.1 to 10. More specifically, it may be 0.2, 0.3, 0.4, 0.5, 0.6, 0.7, 0.8, 0.9, 1, 1.1, 1.2, 1.3, 1.4, 1.5, 2, 2.5, 3, 3.5, 4, 4.5, 5, 5.5, 6, 6.5, 7, 7.5, 8, 8.5, 9, 9.5, etc. A more preferred range is 0.2˜5.

In the above technical solution, the molar ratio of the glycol to Intermediate Compound 3 in step (2) is preferably greater than 1 and less than 2, more preferably 1.1˜1.5. Examples include, but are not limited to: 1.05, 1.1, 1.15, 1.20, 1.25, 1.3, 1.35, 1.4, 1.45, 1.5, 1.55, 1.6, 1.65, 1.7, 1.75, 1.8, 1.85, 1.9, 1.95, etc.

In the above technical solution, the esterification reaction in step (2) is preferably carried out to a degree of esterification of 95˜99%, for example and without limitation: 95.5%, 96%, 96.5%, 97%, 97.5%, 98%, 98.5%, etc.

To achieve this degree of esterification, those skilled in the art may reasonably determine the esterification temperature and time based on practical conditions and equipment capabilities. As a general rule, higher temperatures and longer times improve the degree of esterification.

As a non-limiting example, an esterification temperature in step (2) may be in the range of 150˜250° C. Examples include, but are not limited to: 160° C., 170° C., 180° C., 190° C., 200° C., 210° C., 220° C., 230° C., 240° C., etc. Within this temperature range, when water produced during the esterification reaction is continuously removed, the required degree of esterification can typically be achieved within 0.5˜2 hours even without using a catalyst as in the embodiments. Of course, the esterification catalyst can be applied and using an esterification catalyst can accelerate the reaction.

The esterification reaction in step (2) may be carried out under self-generated pressure or under an inert gas such as nitrogen at a pressure higher than self-generated pressure. Since the reaction is in the liquid phase, pressure does not significantly affect the esterification process.

It is known to those skilled in the art that step (2) and step (i), as esterification reactions, are relatively easy to carry out and may optionally use catalysts. However, the pre-polycondensation in step (ii) and final polycondensation in step (iii) processes involve growing polymer chains, which restricts the activity of reactive groups. Therefore, polymerization catalysts are generally needed. Such catalysts may be added during the esterification stage in step (2) and/or in step (i), allowing them to carry over into the polycondensation stage in step (ii) and step (iii). Alternatively, they may be added directly during pre-polycondensation in step (2) and/or polycondensation in step (3). All of these approaches can achieve comparable technical results without requiring inventive effort.

To improve the dispersion and catalytic uniformity of the catalyst in step (ii) and step (iii), it is preferable to add the catalyst during the esterification stages of step (2) and/or step (i). For comparison purposes, in the embodiments of this invention, the polymerization catalyst is added during step (i).

As for the polymerization catalysts, those known in the art may be used, including but not limited to antimony-based polymerization catalysts (such as antimony trioxide, antimony acetate, and antimony glycolate), titanium-based polymerization catalysts, etc. There are no restrictions on this, and comparable technical effects can all be achieved. In embodiments, all polymerization catalysts used are antimony-based catalysts for comparison purpose only. Dosage of the antimony-based catalysts, calculated as elemental Sb by weight relative to the terephthalic acid used in step (i), is preferably 100˜300 ppmw (for example, but not limited to, 110 ppmw, 120 ppmw, 130 ppmw, 140 ppmw, 150 ppmw, 160 ppmw, 170 ppmw, 180 ppmw, 190 ppmw, 200 ppmw, 210 ppmw, 220ppmw, 230ppmw, 240ppmw, 250ppmw, 260ppmw, 270 ppmw, 280 ppmw, 290 ppmw, etc.). In the embodiments, the antimony catalyst used is antimony glycolate for comparison purpose only.

With respect to the concept of degree of esterification in step (2) and step (i), those skilled in the art understand that degree of esterification is used to monitor the completeness of the esterification reaction. It is defined as the ratio of the total moles of the generated ester groups to the total moles of carboxyl groups in the starting materials. The degree of esterification may be controlled by measuring the weight of water produced and distilled during the reaction. The measurement method of the degree of esterification is obtained by comparing the actual weight of water produced in the esterification reaction with the weight of water produced when the carboxyl group in the assumed reaction raw materials is completely esterified. It is calculated using the following formula:

$$Degree of esterification ( \% ) = ( Weight of Water Actually Produced / Theoretical Weight of Water if All Carboxyl Groups are Esterified ) \times 100 \% .$$

The second technical problem addressed by the present invention is to provide an antibacterial polyester.

To solve the second technical problem described above, the technical solution of the present invention is as follows:

An antibacterial polyester, wherein the antibacterial polyester is obtained by any one of the preparation methods described in the technical solutions to the first technical problem above.

The third technical problem addressed by the present invention is to provide a method for producing an antibacterial fiber.

To solve the third technical problem described above, the technical solution of the present invention is as follows:

A method for producing an antibacterial fiber, comprising the following steps:
(a) blending a fiber-grade PET polyester with the antibacterial polyester described in the technical solution to the second technical problem above, and performing melt spinning to obtain a primary fiber;
(b) thermally drawing the primary fiber.

The key technical feature of the invention lies in the selection of the antibacterial component (or, in other words, the selection of the antibacterial polyester). There are no particular limitations on the specific production process for the antibacterial fiber. A person skilled in the art may reasonably select and adjust among commonly used processes and process conditions to achieve comparable technical effects. The processes and process conditions described below are provided as non-limiting examples.

For example, and without limitation, in step (a), a mass ratio of the antibacterial polyester (based on N content) to the fiber-grade PET polyester may be q:100, where q is greater than 0 and less than or equal to 0.2. More specifically, and without limitation, q may be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, etc. A more preferred range is 0.02˜0.15. For comparison purposes only, the value of q used in the embodiments of the invention is typically 0.04.

For example, and without limitation, in step (a), intrinsic viscosity of the fiber-grade PET polyester is from 0.60 to 0.70 dl/g. Non-limiting examples include: 0.61 dl/g, 0.62 dl/g, 0.63 dl/g, 0.64 dl/g, 0.65 dl/g, 0.66 dl/g, 0.67 dl/g, 0.68 dl/g, 0.69 dl/g, etc. For comparison purposes, the commercial fiber-grade polyethylene terephthalate (abbreviated as commercial fiber-grade PET) used in the embodiments of the invention is SB500 produced by Sinopec Yizheng Chemical Fibre Co., Ltd., which has nominal intrinsic viscosity of 0.67 dl/g and measured intrinsic viscosity of 0.68 dl/g.

For example, and without limitation, the above melt spinning process may be used to produce either filament or staple fibers. The filament may be FDY, POY or DTY made from POY with elasticization. Specifications of the filament may be 50˜300D (for example, but not limited to, 50 D, 60 D, 70 D, 80 D, 90 D, 100 D, 110 D, 120 D, 130 D, 140 D, 150 D, 160 D, 170 D, 180 D, 190 D, 200 D). The length of staple fibers may be 38˜76 mm, with a fineness of 0.5˜3.0 D. Whether filament or staple fibers are produced, regardless of the above specifications or the specific process used, these are not essential features of the present invention, and comparable technical effects can be achieved. For comparison purposes, the embodiments of the invention all use the FDY process to produce filament with a specification of 150 D.

In the above technical solution, a drawing temperature in step (b) is preferably 120˜160° C. For example, and without limitation, the temperature may be 120° C., 125° C., 130° C., 135° C., 140° C., 145° C., 150° C., 155° C., or 160° C. For comparison purposes only, in the embodiments of the present invention, the drawing temperature is 140° C.

In the above technical solution, a draw ratio in step (b) is preferably from 3.0 to 5.0. For example, and without limitation, the draw ratio may be 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. For comparison purposes only, in the embodiments of the present invention, the draw ratio used is 4.0.

The fourth technical problem addressed by the present invention is to provide an antibacterial fiber.

To solve the fourth technical problem described above, the technical solution of the present invention is as follows:

An antibacterial fiber, wherein the antibacterial fiber is obtained by any one of the production methods described in the technical solutions to the third technical problem above.

The fifth technical problem addressed by the present invention is to provide applications of the antibacterial fiber.

To solve the fifth technical problem described above, the technical solution of the present invention is as follows:

The antibacterial fiber described in the technical solution to the fourth technical problem is used in the production of medical surgical article, in particular in the manufacture of antibacterial gauze and/or antibacterial bandages.

Because the antibacterial fiber has antibacterial properties, it can be understood by those skilled in the art that it is suitable for use in the production of medical gauze and bandages. Due to the inherent antibacterial nature of the fiber, using gauze or bandages made from antibacterial fibers helps prevent wound infections. The specific methods of converting antibacterial fibers into gauze or bandages can be selected from known methods in the art, and do not require inventive effort.

The intrinsic viscosity referred to in the present invention is measured according to Method A in Section 5.1.1 of GB/T 14190-2017 (Test Methods for Fiber-grade Polyester (PET) Chips), using a solvent mixture of phenol and 1,1,2,2-tetrachloroethane at a mass ratio of 50:50.

The nitrogen content in the embodiments of the invention is measured by the Kjeldahl method.

The durability of the antibacterial performance of the antibacterial polyester fiber is evaluated in accordance with GB/T 20944.3-2008 (Part 3: Shake Flask Method of the Evaluation of Antibacterial Activity of Textiles). Prior to testing, the fiber product is washed 50 times using Method 10.1.2 specified in the standard. The test bacterium used is Staphylococcus aureus (ATCC6538). The higher the antibacterial rate measured after 50 washes, the better the antibacterial performance and durability of the fiber product.

The present invention is further illustrated by following embodiments.

### DETAILED DESCRIPTION OF THE INVENTION

In the embodiments and comparative examples, the antibacterial rate refers to the rate measured after 50 washing cycles as described above. In this specification, “ppmw” refers to parts per million by weight (1/1,000,000 by weight).

### Embodiment 1

### 1. Synthesis of the Reactive Antibacterial Component

### 1.1. Preparation of Compound Represented by Formula (3)

Under stirring, an aqueous solution of cetyltrimethylammonium chloride (5.6% by weight concentration) was added to an equal weight of aqueous solution of sodium 5- sulfoisophthalate (5.0% by weight concentration). The mixture was stirred for 2 hours and then allowed to stand for phase separation. The resulting precipitate was filtered, washed three times with deionized water (each time using a volume of water equal to that of the filter cake), and dried at 80° C. for 5 hours to obtain the compound represented by Formula (3) in dry form. The nitrogen content of the product was measured to be 2.64% by weight.

### 1.2. Preparation of Reactive Antibacterial Component

Ethylene glycol and the product from step 1.1 were mixed at a molar ratio of 1.3:1 and subjected to an esterification reaction at 180° C. Water produced from the esterification reaction was collected and measured. When a degree of esterification reached 97%, the reactive antibacterial component was obtained. The nitrogen content of this component was measured to be 2.43% by weight.

### 2. Preparation of Antibacterial Polyester

### 2.1. Esterification

Terephthalic acid, antimony glycolate, and ethylene glycol were mixed (a molar ratio of ethylene glycol to terephthalic acid was 1.2; the amount of antimony, calculated as Sb, was 200 ppmw relative to the weight of terephthalic acid). The mixture was subjected to an esterification reaction at 200° C., and water generated was collected and measured. When a degree of esterification reached 97%, Esterified Material I was obtained.

### 2.2. Pre-Polycondensation

The product from step 1.2 (based on nitrogen weight) and Esterified Material I from step 2.1 (based on the amount of terephthalic acid used for its preparation) were mixed at a weight ratio of 1.2:100. The mixture was subjected to polycondensation under an absolute pressure of 500 Pa at 260° C. for 45 minutes.

### 2.3. Final Polycondensation

The product obtained from step 2.2 was subjected to a polycondensation reaction under an absolute pressure of 50 Pa at 280° C. until intrinsic viscosity reached 0.68 dl/g, yielding the antibacterial polyester. The nitrogen content of the resulting polyester was measured to be 0.73% by weight.

### 3. Production of Antibacterial Fiber

### 3.1. Melt Spinning

The antibacterial polyester obtained from step 2.3 (based on nitrogen content) was blended with commercial fiber-grade PET at a weight ratio of 0.040:100 and subjected to melt spinning at 280° C. to produce primary fibers.

### 3.2. Drawing

The primary fibers obtained from melt spinning were thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 89.1%.

### Comparative Embodiment 1

The primary difference from Embodiment 1 is that the compound represented by Formula (3) was added directly during the esterification stage in the preparation of the antibacterial polyester. Specifically:

### 1. Preparation of Compound Represented by Formula (3) (Same as Step 1.1 in Embodiment 1)

Under stirring, an aqueous solution of cetyltrimethylammonium chloride (5.6% by weight concentration) was added to an equal weight of aqueous solution of sodium 5-sulfoisophthalate (5.0% by weight concentration). The mixture was stirred for 2 hours, allowed to stand for phase separation, then filtered. The filter cake was washed three times with water (each time using a volume of water equal to the filter cake), and dried at 80° C. for 5 hours to obtain the dry product of Formula (3). The nitrogen content of the product was measured to be 2.64% by weight.

### 2. Preparation of Antibacterial Polyester

### 2.1. Esterification

Terephthalic acid, the product from step 1, antimony glycolate, and ethylene glycol were mixed as raw materials. A molar ratio of ethylene glycol to the total amount of step 1 product plus terephthalic acid was 1.2. A weight ratio of nitrogen from the step 1 product to terephthalic acid was 1.2:100. The amount of antimony glycolate, calculated as Sb, was 200 ppmw relative to the weight of terephthalic acid. The mixture was subjected to an esterification reaction at 200° C., and water generated was collected and measured. When a degree of esterification reached 97%, Esterified Material I was obtained.

### 2.2. Pre-Polycondensation

The Esterified Material I from step 2.1 was subjected to pre-polycondensation at an absolute pressure of 500 Pa and 260° C. for 45 minutes.

### 2.3. Final Polycondensation

The material from step 2.2 was subjected to polycondensation at an absolute pressure of 50 Pa and 280° C. until intrinsic viscosity reached 0.68 dl/g, yielding the antibacterial polyester.

The nitrogen content of the resulting polyester was measured to be 0.73% by weight.

### 3. Production of Antibacterial Fiber

### 3.1. Melt Spinning

The antibacterial polyester obtained from step 2.3 (based on N content) was blended with commercial fiber-grade PET at a weight ratio of 0.040:100 and subjected to melt spinning at 280° C. to obtain the primary fiber.

### 3.2. Drawing

The primary fiber obtained from melt spinning was thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 76.8%.

### Comparative Embodiment 2

### 1. Synthesis of the Reactive Antibacterial Component

Same as in Embodiment 1, specifically:

### 1.1. Preparation of Compound Represented by Formula (3)

Under stirring, an aqueous solution of cetyltrimethylammonium chloride (5.6% by weight concentration) was added to an equal weight of aqueous solution of sodium 5-sulfoisophthalate (5.0% by weight concentration). The mixture was stirred for 2 hours, allowed to stand for phase separation, then filtered. The filter cake was washed three times with water (each time using a volume of water equal to the volume of the filter cake), and dried at 80° C. for 5 hours to obtain the dry product of Formula (3). The nitrogen content of the product was measured to be 2.64% by weight.

### 1.2. Preparation of Reactive Antibacterial Component

Ethylene glycol and the product from step 1.1 were mixed at a molar ratio of 1.3:1 and subjected to esterification at 180° C. The water generated during the esterification reaction was collected and measured. When a degree of esterification reached 97%, the reactive antibacterial component was obtained. The nitrogen content was measured to be 2.43% by weight.

### 2. Preparation of Antibacterial Polyester

The main difference from Embodiment 1 is that the product from step 1.2 was added during the esterification step of the antibacterial polyester preparation. Specifically:

### 2.1. Esterification

Terephthalic acid, the product from step 1.2, antimony glycolate, and ethylene glycol were mixed as raw materials. A molar ratio of ethylene glycol to terephthalic acid was 1.2. A weight ratio of nitrogen from the step 1.2 product to terephthalic acid was 1.2:100. The amount of antimony glycolate, calculated as Sb, was 200 ppmw relative to the weight of terephthalic acid. The mixture was subjected to esterification at 200° C., and water generated was collected and measured. When a degree of esterification reached 97%, Esterified Material I was obtained.

### 2.2. Pre-Polycondensation

The Esterified Material I from step 2.1 was subjected to pre-polycondensation under an absolute pressure of 500 Pa at 260° C. for 45 minutes.

### 2.3. Final Polycondensation

The product obtained from step 2.2 was subjected to polycondensation under an absolute pressure of 50 Pa at 280° C. until an intrinsic viscosity reached 0.68 dl/g, yielding the antibacterial polyester. The nitrogen content of the polyester was measured to be 0.73% by weight.

### 3. Production of Antibacterial Fiber

### 3.1. Melt Spinning

The antibacterial polyester obtained from step 2.3 (based on N content) was blended with commercial fiber-grade PET at a weight ratio of 0.040:100, and subjected to melt spinning at 280° C. to produce primary fibers.

### 3.2. Drawing

The primary fibers obtained from melt spinning were thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 82.9%.

### Embodiment 2

### 1. Synthesis of the Reactive Antibacterial Component

### 1.1. Preparation of Compound Represented by Formula (3)

Same as step 1.1 in Embodiment 1. Specifically:

Under stirring, an aqueous solution of cetyltrimethylammonium chloride (5.6% by weight concentration) was added to an equal weight of aqueous solution of sodium 5-sulfoisophthalate (5.0% by weight). The mixture was stirred for 2 hours, allowed to stand for phase separation, then filtered. The filter cake was washed three times with water (each time using a volume of water equal to the volume of the filter cake), and dried at 80° C. for 5 hours to obtain the dry product of Formula (3). The nitrogen content of the product was measured to be 2.64% by weight.

### 1.2. Preparation of Reactive Antibacterial Component

The main difference from step 1.2 in Embodiment 1 is that 1,4-butanediol was used as the glycol. Specifically:

1,4-butanediol and the product from step 1.1 were mixed at a molar ratio of 1.3:1 and subjected to esterification at 180° C. The water generated during the reaction was collected and measured. When a degree of esterification reached 97%, the reactive antibacterial component was obtained. The nitrogen content of the product was measured to be 2.27% by weight.

### 2. Preparation of Antibacterial Polyester

### 2.1. Esterification

Terephthalic acid, antimony glycolate, and ethylene glycol were mixed as raw materials (the molar ratio of ethylene glycol to terephthalic acid was 1.2; the amount of antimony glycolate, calculated as Sb, was 200 ppmw relative to the weight of terephthalic acid). The mixture was subjected to esterification at 200° C., and water generated during the reaction was collected and measured. When a degree of esterification reached 97%, Esterified Material I was obtained.

### 2.2. Pre-Polycondensation

The product from step 1.2 (based on nitrogen content) and Esterified Material I from step 2.1 (based on the weight of terephthalic acid required for its preparation) were mixed at a weight ratio of 1.2:100. The mixture was subjected to pre-polycondensation at an absolute pressure of 500 Pa and 260° C. for 45 minutes.

### 2.3. Final Polycondensation

The material obtained from step 2.2 was subjected to final polycondensation at an absolute pressure of 50 Pa and 280° C. until intrinsic viscosity reached 0.68 dl/g, yielding the antibacterial polyester. The nitrogen content of the polyester was measured to be 0.72% by weight.

### 3. Production of Antibacterial Fiber

### 3.1. Melt Spinning

The antibacterial polyester obtained from step 2.3 (based on N content) was blended with commercial fiber-grade PET at a weight ratio of 0.040:100 and subjected to melt spinning at 280° C. to obtain primary fibers.

### 3.2. Drawing

The primary fibers obtained by melt spinning were thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 87.6%.

### Embodiment 3

### 1. Synthesis of the Reactive Antibacterial Component

### 1.1. Preparation of Compound Represented by Formula (3)

Same as step 1.1 in Embodiment 1. Specifically:

Under stirring, an aqueous solution of cetyltrimethylammonium chloride (5.6% by weight concentration) was added to an equal weight of aqueous solution of sodium 5-sulfoisophthalate (5.0% by weight concentration). The mixture was stirred for 2 hours, allowed to stand for phase separation, then filtered. The filter cake was washed three times with water (each time using a volume of water equal to the volume of the cake), and dried at 80° C. for 5 hours to obtain the dry product of Formula (3). The nitrogen content of the product was measured to be 2.64% by weight.

### 1.2. Preparation of Reactive Antibacterial Component

The main difference from step 1.2 in Embodiment 1 is that PEG 400 was used as the glycol. Specifically:

PEG 400 and the product from step 1.1 were mixed at a molar ratio of 1.3:1 and subjected to esterification at 180° C. The water generated during the reaction was collected and measured. When a degree of esterification reached 97%, the reactive antibacterial component was obtained. The nitrogen content of the product was measured to be 1.37% by weight.

### 2. Preparation of Antibacterial Polyester

### 2.1. Esterification

Terephthalic acid, antimony glycolate, and ethylene glycol were mixed as raw materials (a molar ratio of ethylene glycol to terephthalic acid was 1.2; the amount of antimony glycolate, calculated as Sb, was 200 ppmw relative to the weight of terephthalic acid). The mixture was subjected to esterification at 200° C., and water generated during the reaction was collected and measured. When a degree of esterification reached 97%, Esterified Material I was obtained.

### 2.2. Pre-Polycondensation

The product from step 1.2 (based on nitrogen content) and Esterified Material I from step 2.1 (based on the weight of terephthalic acid required for its preparation) were mixed at a weight ratio of 1.2:100. The mixture was subjected to pre-polycondensation at an absolute pressure of 500 Pa and 260° C. for 45 minutes.

### 2.3. Final Polycondensation

The material obtained from step 2.2 was subjected to polycondensation at an absolute pressure of 50 Pa and 280° C. until intrinsic viscosity reached 0.68 dl/g, yielding the antibacterial polyester. The nitrogen content of the polyester was measured to be 0.59% by weight.

### 3. Production of Antibacterial Fiber

### 3.1. Melt Spinning

The antibacterial polyester obtained from step 2.3 (based on N content) was blended with commercial fiber-grade PET at a weight ratio of 0.040:100 and subjected to melt spinning at 280° C. to obtain primary fibers.

### 3.2. Drawing

The primary fibers obtained by melt spinning were thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 90.9%.

### Embodiment 4

### 1. Synthesis of the Reactive Antibacterial Component

### 1.1. Preparation of Compound Represented by Formula (3)

Same as step 1.1 in Embodiment 1. Specifically:

Under stirring, an aqueous solution of cetyltrimethylammonium chloride (5.6% by weight concentration) was added to an equal weight of aqueous solution of sodium 5-sulfoisophthalate (5.0% by weight concentration). The mixture was stirred for 2 hours, allowed to stand for phase separation, then filtered. The filter cake was washed three times with water (each time using a volume of water equal to the volume of the filter cake), and dried at 80° C. for 5 hours to obtain the dry product of Formula (3). The nitrogen content of the product was measured to be 2.64% by weight.

### 1.2. Preparation of Reactive Antibacterial Component

The main difference from step 1.2 in Embodiment 1 is that 1,4-cyclohexanedimethanol was used as the glycol. Specifically:

1,4-cyclohexanedimethanol and the product from step 1.1 were mixed at a molar ratio of 1.3:1 and subjected to esterification at 180° C. Water generated during the reaction was collected and measured. When a degree of esterification reached 97%, the reactive antibacterial component was obtained. The nitrogen content of the product was measured to be 2.03% by weight.

### 2. Preparation of Antibacterial Polyester

### 2.1. Esterification

Terephthalic acid, antimony glycolate, and ethylene glycol were mixed as raw materials (a molar ratio of ethylene glycol to terephthalic acid was 1.2; the amount of antimony glycolate, calculated as Sb, was 200 ppmw relative to the weight of terephthalic acid). The mixture was subjected to esterification at 200° C., and water generated during the reaction was collected and measured. When a degree of esterification reached 97%, Esterified Material I was obtained.

### 2.2. Pre-Polycondensation

The product from step 1.2 (based on nitrogen content) and Esterified Material I from step 2.1 (based on the weight of terephthalic acid required for its preparation) were mixed at a weight ratio of 1.2:100. The mixture was subjected to pre-polycondensation at an absolute pressure of 500 Pa and 260° C. for 45 minutes.

### 2.3. Final Polycondensation

The material obtained from step 2.2 was subjected to polycondensation at an absolute pressure of 50 Pa and 280° C. until intrinsic viscosity reached 0.68 dl/g, yielding the antibacterial polyester. The nitrogen content of the polyester was measured to be 0.69% by weight.

### 3. Production of Antibacterial Fiber

### 3.1. Melt Spinning

The antibacterial polyester obtained from step 2.3 (based on N content) was blended with commercial fiber-grade PET at a weight ratio of 0.040:100 and subjected to melt spinning at 280° C. to obtain primary fibers.

### 3.2. Drawing

The primary fibers obtained by melt spinning were thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 95.3%.

### Embodiment 5

### 1. Synthesis of the Reactive Antibacterial Component

### 1.1. Preparation of Compound Represented by Formula (3)

Same as step 1.1 in Embodiment 1. Specifically:

Under stirring, an aqueous solution of cetyltrimethylammonium chloride (5.6% by weight concentration) was added to an equal weight of aqueous solution of sodium 5-sulfoisophthalate (5.0% by weight concentration). The mixture was stirred for 2 hours, allowed to stand for phase separation, then filtered. The filter cake was washed three times with water (each time using a volume of water equal to the volume of the filter cake), and dried at 80° C. for 5 hours to obtain the dry product of Formula (3). The nitrogen content of the product was measured to be 2.64% by weight.

### 1.2. Preparation of Reactive Antibacterial Component

The main difference from step 1.2 in Embodiment 1 is that a mixture of ethylene glycol and 1,4-cyclohexanedimethanol was used as the glycol. Specifically:

Ethylene glycol, 1,4-cyclohexanedimethanol, and the product from step 1.1 were mixed (the molar ratio of ethylene glycol to 1,4-cyclohexanedimethanol was 1:1; the total molar ratio of the glycols to the compound from step 1.1 was 1.3:1). The mixture was subjected to esterification at 180° C. Water generated during the reaction was collected and measured. When a degree of esterification reached 97%, the reactive antibacterial component was obtained. The nitrogen content of the product was measured to be 2.21% by weight.

### 2. Preparation of Antibacterial Polyester

### 2.1. Esterification

Terephthalic acid, antimony glycolate, and ethylene glycol were mixed as raw materials (a molar ratio of ethylene glycol to terephthalic acid was 1.2; the amount of antimony glycolate, calculated as Sb, was 200 ppmw relative to the weight of terephthalic acid). The mixture was subjected to esterification at 200° C., and water generated during the reaction was collected and measured. When a degree of esterification reached 97%, Esterified Material I was obtained.

### 2.2. Pre-Polycondensation

The product from step 1.2 (based on nitrogen content) and Esterified Material I from step 2.1 (based on the weight of terephthalic acid required for its preparation) were mixed at a weight ratio of 1.2:100. The mixture was subjected to pre-polycondensation at an absolute pressure of 500 Pa and 260° C. for 45 minutes.

### 2.3. Final Polycondensation

The material obtained from step 2.2 was subjected to polycondensation at an absolute pressure of 50 Pa and 280° C. until intrinsic viscosity reached 0.68 dl/g, yielding the antibacterial polyester. The nitrogen content of the polyester was measured to be 0.71% by weight.

### 3. Production of Antibacterial Fiber

### 3.1. Melt Spinning

The antibacterial polyester obtained from step 2.3 (based on N content) was blended with commercial fiber-grade PET at a weight ratio of 0.040:100 and subjected to melt spinning at 280° C. to obtain primary fibers.

### 3.2. Drawing

The primary fibers obtained by melt spinning were thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 99.7%.

### Embodiment 6

### 1. Synthesis of the Reactive Antibacterial Component

### 1.1. Preparation of Compound Represented by Formula (3)

Same as step 1.1 in Embodiment 1. Specifically:

Under stirring, an aqueous solution of cetyltrimethylammonium chloride (5.6% by weight concentration) was added to an equal weight of aqueous solution of sodium 5-sulfoisophthalate (5.0% by weight concentration). The mixture was stirred for 2 hours, allowed to stand for phase separation, then filtered. The filter cake was washed three times with water (each time using a volume of water equal to the volume of the cake), and dried at 80° C. for 5 hours to obtain the dry product of Formula (3). The nitrogen content of the product was measured to be 2.64% by weight.

### 1.2. Preparation of Reactive Antibacterial Component

The main difference from step 1.2 in Embodiment 1 is that a mixture of ethylene glycol and 1,4-cyclohexanedimethanol was used as the glycol. Specifically:

Ethylene glycol, 1,4-cyclohexanedimethanol, and the product from step 1.1 were mixed as reaction raw materials (the molar ratio of ethylene glycol to 1,4-cyclohexanedimethanol was 4:1; the total molar ratio of the diols to the product from step 1.1 was 1.3:1). The mixture was subjected to esterification at 180° C. The water generated during the reaction was collected and measured. When a degree of esterification reached 97%, the reactive antibacterial component was obtained. The nitrogen content of the product was measured to be 2.34% by weight.

### 2. Preparation of Antibacterial Polyester

### 2.1. Esterification

Terephthalic acid, antimony glycolate, and ethylene glycol were mixed as raw materials (a molar ratio of ethylene glycol to terephthalic acid was 1.2; the amount of antimony glycolate, calculated as Sb, was 200 ppmw relative to the weight of terephthalic acid). The mixture was subjected to esterification at 200° C., and water generated during the reaction was collected and measured. When a degree of esterification reached 97%, Esterified Material I was obtained.

### 2.2. Pre-Polycondensation

The product from step 1.2 (based on nitrogen content) and Esterified Material I from step 2.1 (based on the weight of terephthalic acid required for its preparation) were mixed at a weight ratio of 1.2:100. The mixture was subjected to pre-polycondensation at an absolute pressure of 500 Pa and 260° C. for 45 minutes.

### 2.3. Final Polycondensation

The material obtained from step 2.2 was subjected to polycondensation at an absolute pressure of 50 Pa and 280° C. until intrinsic viscosity reached 0.68 dl/g, yielding the antibacterial polyester. The nitrogen content of the polyester was measured to be 0.72% by weight.

### 3. Production of Antibacterial Fiber

### 3.1. Melt Spinning

The antibacterial polyester obtained from step 2.3 (based on N content) was blended with commercial fiber-grade PET at a weight ratio of 0.040:100 and subjected to melt spinning at 280° C. to obtain primary fibers.

### 3.2. Drawing

The primary fibers obtained by melt spinning were thermally drawn at a draw ratio of 4.0 and a temperature of 140° C. The drawn fibers were then spun into FDY fibers with a specification of 150 D.

### 4. Testing of Antibacterial Polyester Fiber

An antibacterial rate was measured to be 98.5%.

What is claimed is:

1. A method for preparing an antibacterial polyester, comprising:
step (i) carrying out an esterification reaction between a diol and a dibasic acid to obtain Esterified Material I;
step (ii) mixing the Esterified Material I with a reactive antibacterial component, and conducting a pre-polycondensation reaction to obtain a pre-polycondensate;
step (iii) performing a final polycondensation reaction on the pre-polycondensate to obtain the antibacterial polyester;
wherein the reactive antibacterial component is obtained by esterification of a compound represented by Formula (3) and a glycol: 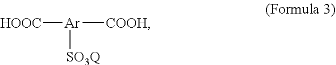
wherein Q represents a quaternary ammonium group containing a long-chain hydrocarbon group containing 6 to 20 carbon atoms; Ar represents an aromatic ring;
and the glycol comprises 1,4-cyclohexanedimethanol.

2. The method according to claim 1, wherein the glycol comprises a mixed glycol of ethylene glycol and 1,4-cyclohexanedimethanol.

3. The method according to claim 2, wherein a molar ratio of the ethylene glycol to 1,4-cyclohexanedimethanol is from 0.1 to 10.

4. The method according to claim 3, wherein the molar ratio of ethylene glycol to 1,4-cyclohexanedimethanol is from 0.2 to 5.

5. The method according to claim 1, wherein Ar is a phenyl ring or a naphthyl ring.

6. The method according to claim 1, wherein in step (i):
the diol comprises at least one selected from a group consisting of ethylene glycol, 1,3-propanediol, 1,4-butanediol, 1,5-pentanediol, and 1,4-cyclohexanedimethanol; and/or
the dibasic acid comprises at least one selected from a group consisting of terephthalic acid, succinic acid, adipic acid, isophthalic acid, and furan dicarboxylic acid; and/or a molar ratio of diol to dibasic acid is from 1.1 to 1.5; and/or the esterification reaction proceeds until a degree of esterification of 95˜99% is achieved; and/or the reaction temperature is from 150° C. to 250° C.

7. The method according to claim 1, wherein in step (ii): a reaction pressure is from 400 to 600 Pa;
and/or a reaction temperature is from 255° C. to 265° C.; and/or a reaction time is from 30 to 60 minutes.

8. The method according to claim 1, wherein in step (iii): a reaction pressure is less than or equal to 100 Pa; and/or the reaction temperature is from 270° C. to 285° C.; and/or the final polycondensation proceeds until the intrinsic viscosity reaches 0.60˜0.75 dL/g.

9. The method according to claim 1, wherein the reactive antibacterial component is synthesized by:
(1) subjecting Compound 1 and Compound 2 to an ion exchange reaction in a solvent to obtain Intermediate Compound 3,
wherein Compound 1 has the structure represented by Formula (1): 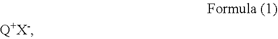
Compound 2 has the structure represented by Formula (2): 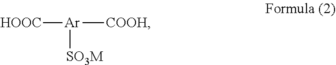
and Intermediate Compound 3 has the structure represented by Formula (3);
wherein X is Cl or Br; M is an alkali metal;
(2) esterifying Intermediate Compound 3 with a glycol to obtain the reactive antibacterial component.

10. The method according to claim 9, wherein the solvent in step (1) is water.

11. The method according to claim 9, wherein: Compound 1 has the structure represented by Formula (1a): 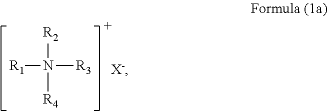
R₁ is a long-chain hydrocarbon, containing 6˜20 carbon atoms;
R₂˜R₄ are short-chain hydrocarbons;
and/or
wherein Compound 2 has the structure represented by Formula (2a): 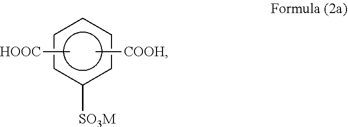
and/or the molar ratio of glycol to Intermediate Compound 3 in step (2) is greater than 1 and less than 2;
and/or the esterification in step (2) proceeds until a degree of esterification reaches 95˜99%;
and/or the esterification temperature in step (2) is from 150° C. to 250° C.

12. The method according to claim 11, wherein: R₂˜R₄ are independently C₁˜C₂ alkyl groups; and/or the molar ratio of glycol to Intermediate Compound 3 in step (2) is from 1.1 to 1.5.

13. An antibacterial polyester obtained by the method according to claim 1.

14. An antibacterial polyester obtained by the method according to claim 2.

15. An antibacterial polyester obtained by the method according to claim 9.

16. A method for producing an antibacterial fiber, comprising:
(a) blending a fiber-grade PET polyester with the antibacterial polyester of claim 13, and performing melt spinning to obtain a primary fiber;
(b) thermally drawing the primary fiber.

17. The method according to claim 16, wherein: in step (a), the antibacterial polyester is blended such that the N content in the blend corresponds to a weight ratio of b: 100 relative to the fiber-grade PET polyester, wherein b is greater than 0 and less than or equal to 0.2; and/or in step (b), the drawing temperature is from 120° C. to 160° C.; and/or in step (b), the draw ratio is from 3.0 to 5.0.

18. An antibacterial fiber obtained by the method according to claim 16.

19. Use of the antibacterial fiber of claim 18 in the manufacture of medical surgical article.

20. Use of the antibacterial fiber of claim 18 in the manufacture of antibacterial gauze and/or antibacterial bandages.
